(19) United States
(12) Patent Application Publication
(10) Pub. No.: US 2017/0058302 A1
(43) Pub. Date: Mar. 2, 2017

(54) METHOD FOR EFFICIENT ENZYMATIC HYDROLYSIS OF LIGNOCELLULOSIC MATERIALS

(71) Applicant: PRAJ INDUSTRIES LIMITED, Pune (IN)

(72) Inventors: Sasisanker PADMANABHAN, Pune (IN); Siddhartha PAL, Pune (IN); Shereena Panakal JOY, Pune (IN)

(21) Appl. No.: 15/121,121

(22) PCT Filed: Apr. 6, 2015
(86) PCT No.: PCT/IN2015/000159

§ 371 Date: Aug. 24, 2016

(30) Foreign Application Priority Data
Apr. 7, 2014 (IN) 1296/MUM/2014

(51) Int. Cl. C12P 7/10

(52) U.S. Cl. CPC C12P 7/10; C12P 2203/00

(57) ABSTRACT

The invention relates to a method for efficient enzymatic hydrolysis of lignocellulosic materials and more particularly, it relates to efficient enzymatic hydrolysis of cellulosic part of lignocellulosic materials like corncob, corn stover, sugarcane/beet bagasse or any similar lignocellulosic materials.

### FIELD OF INVENTION

The invention relates to a method for efficient enzymatic hydrolysis of lignocellulosic materials and more particularly, it relates to efficient enzymatic hydrolysis of cellulosic part of lignocellulosic materials like corncob, corn stover, sugarcane/beet bagasse or any similar lignocellulosic materials to prepare ethanol or other bio-chemicals.

### BACKGROUND

Due to the future limitations on the availability of fossil fuels particularly crude oil, the use of alternate fuels such as ethanol in motor vehicles is promoted worldwide. Ethanol is primarily produced by microbial fermentation of sugars. The three main groups of raw materials for production of ethanol by fermentation are sugars, starch, and lignocelluloses. A disadvantage of using the sugar/starch-based materials for ethanol production is that many of these raw materials are considered a human food resource and will therefore be too expensive to use for fuel ethanol production. A disadvantage of using the lignocellulose-based materials for ethanol production is that the hydrolysis cost is high due to high costs of energy and hydrolytic enzymes.

The largest potential feedstock for ethanol is lignocellulosic materials, (LCM), which includes materials such as agricultural residues (corn stover, crop straws, husks and bagasse), herbaceous crops (alfalfa, switchgrass), short rotation woody crops, forestry residues, waste paper and other wastes (municipal and industrial). Bioethanol production from these feedstocks could be an attractive alternative for disposal of these residues. Lignocellulosic feedstocks do not interfere with food security and are important in terms of energy security reason as well as environmental concern. It is mostly used inefficiently as an energy source or fed to animals; however, a large part is wasted as such without any use.

LCM constitutes a major portion of plant dry matter and has three major components such as cellulose, hemicellulose and lignin. Lignin and hemicellulose can form lignin-carbohydrate bonds, which result in complexes that provide a hydrolysis-resistant protecting sheet around the cellulose.

In the production of bio-ethanol from lignocellulosic biomass, dilute acid treatment is the most preferred pre-treatment as it provides advantages of low cost of acids, ease of unit operation and good separation of C5 sugars. However, the disadvantage of this method is that the C6 enzymatic hydrolysis is low (maximum 50%). This is because of presence of soluble inhibitors and lignin in the substrate. Lignin present in the cake/slurry irreversibly binds to the enzyme thus lowering the performance. Therefore, there is a challenge to overcome the lignin inhibition without loss of sugars.

There are methods, which enhance enzymatic hydrolysis of acid treated biomass slurry/cake by adding surfactants or excess enzymes. In addition, there are methods of over-liming and detoxification using resins and charcoals to enhance enzymatic hydrolysis. Nevertheless, these methods are expensive and may result in delignification and loss of C5 sugars like xylose. Therefore, it is desirable to provide an improved and economical method to reduce enzyme-lignin hindrance without the loss of lignin and sugars. This is achieved by introducing a new improved method of present invention to enhance enzymatic hydrolysis without loss of lignin and sugars.

### BRIEF DESCRIPTION OF THE INVENTION

In one embodiment of the invention, lignocellulosic material is used as a feedstock and its mixture is prepared, in water. Then, the mixture is pre-treated with one or more acids at a desired temperature for a desired time period to obtain a first stream. The pH of the first stream is adjusted with a base or a mixture of bases to obtain a second stream. Said second stream is treated with sodium sulphite at a desired temperature for a desired time period to obtain a third stream. Then, said third stream is contacted with one or more of cellulolytic enzymes at desired temperature for a desired time period to obtain a sugar rich final stream. Said final stream is further subjected to fermenting yeast to obtain ethanol.

### DETAILED DESCRIPTION OF THE INVENTION

Present invention discloses an improved process for enzymatic hydrolysis of cellulosic part of lignocellulosic materials like corncob, corn stover, sugarcane/beet bagasse or any similar lignocellulosic materials.

In one embodiment of the disclosed invention, lignocellulosic material is used as a feedstock and is further subjected to mechanical shearing for size reduction to form particulate material. This particulate material is soaked in water to form slurry and introduced continuously into a plug-screw type hydrolyser. In the hydrolyser, said slurry is mixed with an admixture of organic acid [such as oxalic acid] and mineral acid [such as sulphuric or and phosphoric acid]. The resultant reaction is allowed to take place at temperature of about 140° C. to about 210° C. and at pressure of about 3.5 bar (absolute) to about 23 bar (absolute) for a period of about 5 minutes to about 120 minutes. This results in formation of a first stream. The pH of said first stream is adjusted between about 4 and about 5 with a base such as magnesium oxide or sodium hydroxide or a mixture thereof, to form a second stream. Next, sodium sulphite [or sodium carbonate] is added to the second stream and reaction is allowed to take place at temperature of about 60° C. to about 80° C. for a period 100 of about 10 minutes to about 60 minutes to form a third stream. In this slurry, cellulose is present in insoluble solids. The sulphite treated slurry is subjected to enzymatic hydrolysis with one or more of cellulase and hemicellulase at temperature of about 40° C. to about 80° C. for a period of about 36 hours to about 120 hours to form a final stream. The enzymatic hydrolysis efficiency obtained at the end of hydrolysis was found to have increased up to about 15% of percent points after treatment by sodium sulphite over the efficiency obtained without the use of sodium sulphite.

Examples provided below give wider utility of the invention without any limitations as to the variations that may be appreciated by a person skilled in the art. A non-limiting summary of various experimental results is given in the examples, which demonstrate the advantageous and novel aspects of the process of using LCM for the preparation of ethanol.

### EXAMPLE 1

A batch of about 130 Kg of bagasse having total solids of about 92% by weight, cellulose of about 36% by weight, hemicelluloses of about 21% by weight and lignin of about 19% by weight was used as a feedstock. It was subjected to mechanical shearing for size reduction to less than 40 mm particles affording about 120 Kg of the particulate material. This particulate material was soaked in water for about 30 min. Then about 400 Kg slurry containing about 30% by weight total solids was prepared and continuously introduced into a plug screw type hydrolyser. Here the slurry was mixed with about 160 litres of the admixture of oxalic and sulphuric acids. This admixture of mixed acids contained about 1.0% by weight oxalic acid and about 1.5% by weight sulphuric acid on dry biomass weight basis [total 2.5% acid on dry biomass weight basis]. The resultant reaction mixture was then subjected to hydrolysis in a hydrolyser at a temperature of about 150° C. and pressure of about 5.5 bar [absolute] for a period of about 24 minutes at pH of about 1.2. At the end of this pre-treatment final slurry of about 560 Kg contained about 20% of total solids with about 13% of total insoluble solids. This slurry was then diluted with water to form diluted slurry containing about 16.1% of total solids with about 10.5% of total insoluble solids. The pH of this diluted slurry was about 1.4. In first step,. about 3.5 Kg of NaOH was added to said diluted slurry to achieve the pH of about 5. In this slurry, cellulose form about 55% of total insoluble solids. Then this sulphite treated slurry was subject to enzymatic hydrolysis by addition of about 30 mg of mix of cellulases per 1 g of cellulose present in the slurry. The enzymatic hydrolysis efficiency obtained at the end of 120 h of hydrolysis at 50° C. was about 48% of theoretical maximum possible.

### EXAMPLE 2

A batch of about 130 Kg of bagasse having total solids of about 92% by weight, cellulose of about 36% by weight, hemicelluloses of about 21% by weight and lignin of about 19% by weight was used as a feedstock. It was subjected to mechanical shearing for size reduction to less than 40 mm particles affording about 120 Kg of the particulate material. This particulate material was soaked in water for about 30 min. Then about 400 Kg slurry containing about 30% by weight total solids was prepared and continuously introduced into a plug screw type hydrolyser. Here the slurry was mixed with about 160 litres of the admixture of oxalic and sulphuric acids. This admixture of mixed acids contained about 1.0% by weight oxalic acid and about 1.5% by weight sulphuric acid on dry biomass weight basis [total 2.5% acid on dry biomass weight basis]. The resultant reaction mixture was then subjected to hydrolysis in a hydrolyser at a temperature of about 150° C. and pressure of about 5.5 bar [absolute] for a period of about 24 minutes at pH of about 1.2. At the end of this pre-treatment final slurry of about 560 Kg contained about 20% of total solids with about 13% of total insoluble solids. This slurry was then diluted with water to form diluted slurry containing about 17.1% of total solids with about 11.1% of total insoluble solids. The pH of this diluted slurry was about 1.4. In first step, about 3.5 Kg of magnesium oxide was added to said diluted slurry to achieve the pH of about 5.5. In this slurry, cellulose form about 55% of total insoluble solids. Then this slurry was subject to enzymatic hydrolysis by addition of about 30 mg of mix of cellulases per 1 g of cellulose present in the slurry. The enzymatic hydrolysis efficiency obtained at the end of 120 h of hydrolysis at 50° C. was about 50% of theoretical maximum possible.

### EXAMPLE 3

A batch of about 130 Kg of bagasse having total solids of about 92% by weight, cellulose of about 36% by weight, hemicelluloses of about 21% by weight and lignin of about 19% by weight was used as a feedstock. It was subjected to mechanical shearing for size reduction to less than 40 mm particles affording about 120 Kg of the particulate material. This particulate material was soaked in water for about 30 min. Then about 400 Kg slurry containing about 30% by weight total solids was prepared and continuously introduced into a plug screw type hydrolyser. Here the slurry was mixed with about 160 litres of the admixture of oxalic and sulphuric acids. This admixture of mixed acids contained about 1.0% by weight oxalic acid and about 1.5% by weight sulphuric acid on dry biomass weight basis [total 2.5% acid on dry biomass weight basis]. The resultant reaction mixture was then subjected to hydrolysis in a hydrolyser at a temperature of about 150° C. and pressure of about 5.5 bar [absolute] for a period of about 24 minutes at pH of about 1.2. At the end of this pre-treatment final slurry of about 560 Kg contained about 20% of total solids with about 13% of total insoluble solids. This slurry was then diluted with water to form diluted slurry containing about 16.4% of total solids with about 11.2% of total insoluble solids. The pH of this diluted slurry was about 1.4. In first step, about 3 Kg of magnesium oxide was added to said diluted slurry to achieve the pH of about 4.5. In second step, about 1.5 Kg of sodium sulphite was added at this stage the pH of was about 5.5. After these additions, the slurry was allowed to stand at about 70° C. for about 30 min for effect of sulphite to take place on the insoluble solids present in the slurry. In this slurry, cellulose form about 55% of total insoluble solids. Then this sulphite treated slurry was subject to enzymatic hydrolysis by addition of. about 30 mg of mix of cellulases per 1 g of cellulose present in the slurry. The enzymatic hydrolysis efficiency obtained at the end of 120 h of hydrolysis at 50° C. was about 58.7% of theoretical maximum possible.

### EXAMPLE 4

A batch of about 130 Kg of bagasse having total solids of about 92% by weight, cellulose of about 36% by weight, hemicelluloses of about 21% by weight and lignin of about 19% by weight was used as a feedstock. It was subjected to mechanical shearing for size reduction to less than 40 mm particles affording about 120 Kg of the particulate material. This particulate material was soaked in water for about 30 min. Then about 400 Kg slurry containing about 30% by weight total solids was prepared and continuously introduced into a plug screw type hydrolyser. Here the slurry was mixed with about 160 litres of the admixture of oxalic and sulphuric acids. This admixture of mixed acids contained about 1.0% by weight oxalic acid and about 1.5% by weight sulphuric acid on dry biomass weight basis [total 2.5% acid on dry biomass weight basis]. The resultant reaction mixture was then subjected to hydrolysis in a hydrolyser at a temperature of about 150° C. and pressure of about 5.5 bar [absolute] for a period of about 24 minutes at pH of about 1.2. At the end of this pre-treatment final slurry of about 560 Kg contained about 20% of total solids with about 13% of total insoluble solids. This slurry was then diluted with water to form diluted slurry containing about 17.2% of total solids with about 10.75% of total insoluble solids. The pH of this diluted slurry was about 1.4. In first step, about 2.8 Kg of magnesium oxide was added to said diluted slurry to achieve the pH of about 4.5. In second step, about 2.8 Kg of sodium sulphite was added, at this stage the pH of about 5.5. After these additions, the slurry was allowed to stand at about 70° C. for about 30 min for effect of sulphite to take place on the insoluble solids present in the slurry. In this slurry, cellulose form about 55% of total insoluble solids. Then this sulphite treated slurry was subject to enzymatic hydrolysis by addition of about 30 mg of mix of cellulases per 1 g of cellulose present in the slurry. The enzymatic hydrolysis efficiency obtained at the end of 120 h of hydrolysis at 50° C. was about 63.3% of theoretical maximum possible.

### EXAMPLE 5

A batch of about 130 Kg of bagasse having total solids of about 92% by weight, cellulose of about 36% by weight, hemicelluloses of about 21% by weight and lignin of about 19% by weight was used as a feedstock. It was subjected to mechanical shearing for size reduction to less than 40 mm particles affording about 120 Kg of the particulate material. This particulate material was soaked in water for about 30 min. Then about 400 Kg slurry containing about 30% by weight total solids was prepared and continuously introduced into a plug screw type hydrolyser. Here the slurry was mixed with about 160 litres of the admixture of oxalic and sulphuric acids. This admixture of mixed acids contained about 1.0% by weight oxalic acid and about 1.5% by weight sulphuric acid on dry biomass weight basis [total 2.5% acid on dry biomass weight basis]. The resultant reaction mixture was then subjected to hydrolysis in a hydrolyser at a temperature of about 150° C. and pressure of about 5.5 bar [absolute] for a period of about 24 minutes at pH of about 1.2. At the end of this pre-treatment final slurry of about 560 Kg contained about 20% of total solids with about 13% of total insoluble solids. This slurry was then diluted with water to form diluted slurry containing about 16.5% of total solids with about 11.5% of total insoluble solids. The pH of this diluted slurry was about 1.4. In first step, about 3.1 Kg of magnesium oxide was added to said diluted slurry to achieve the pH of about 4.5. In second step, about 3.1 Kg of sodium carbonate was added, at this stage the pH of about 5.5. After these additions, the slurry was allowed to stand at about 70° C. for about 30 min for effect of carbonate to take place on the insoluble solids present in the slurry. In this slurry, cellulose form about 55% of total insoluble solids. Then this carbonate treated slurry was subject to enzymatic hydrolysis by addition of about 30 mg of mix of cellulases per 1 g of cellulose present in the slurry. The enzymatic hydrolysis efficiency obtained at the end of 120 h of hydrolysis at 50° C. was about 55.55% of theoretical maximum possible.

### EXAMPLE 6

As listed in TABLE 1 of enzymatic hydrolysis after treatment with sodium sulphite of acid-treated bagasse shows remarked increase in the efficiency of Conversion of cellulose to glucose by the enzymes at various dosages. Herein about 4% sodium sulphite by weight in the presence of about 4% magnesium oxide by weight was used. Conversion efficiencies were measured for low and high enzyme dosages and controls were used for the comparison to show the pronounced effect of sodium sulphite on the substrate.

TABLE 1
enzymatic hydrolysis on sodium sulphite treated acid-
treated bagasse at different enzyme dosages.

| Total | | Total insoluble | | C6 Conversion | | Enzyme |
|---|---|---|---|---|---|---|
| solids (%) | | solids (%) | | efficiency (%) | | dose |
| | With | | With | | With | mg/g of |
| Control | sulphite | Control | sulphite | Control | sulphite | cellulose |
| 16.4 | 16.9 | 9.75 | 9.95 | 35.55 | 44.67 | 20 |
| | | | | 47.86 | 57.75 | 30 |
| | | | | 65.11 | 72.32 | 60 |
| | | | | 72.59 | 78.71 | 90 |

### EXAMPLE 7

As listed in TABLE 1 of enzymatic hydrolysis after the treatment with sodium sulphite of acid-treated corncob shows remarked increase in the efficiency of conversion of cellulose to glucose by the enzymes at various dosages. Herein about 2% sodium sulphite by weight in the presence of about 4% magnesium oxide by weight was used. Conversion efficiencies were measured for low and high enzyme dosages and controls were used for the comparison to show the pronounced effect of sodium sulphite on the substrate.

TABLE 1
enzymatic hydrolysis on sodium sulphite treated acid-
treated corncob at different enzyme dosages.

| Total | | Total insoluble | | C6 Conversion | | Enzyme |
|---|---|---|---|---|---|---|
| solids (%) | | solids (%) | | efficiency (%) | | dose |
| | With | | With | | With | mg/g of |
| Control | sulphite | Control | sulphite | Control | sulphite | cellulose |
| 17.2 | 16.5 | 9.75 | 8.72 | 41.61 | 49.21 | 10 |
| | | | | 65.74 | 74.84 | 20 |
| | | | | 74.71 | 81.25 | 40 |
| | | | | 79.5 | 83.67 | 60 |

### EXAMPLE 8

A batch of about 130 kg bagasse having total solids around 92% by weight, of which cellulose is 36% by weight, hemicelluloses 21% by weight and insoluble lignin 19 by weight was used as a feedstock. It was subjected to mechanical shearing for size reduction to less than 40 mm particles affording about 120 kg dry particulate material. This particulate material was soaked in water for about 30 min. Then about 400 kg of slurry containing about 30% by weight total solids was prepared and continuously introduced into a plug-screw hydrolyser. Here the slurry was mixed with 160 L of the admixture of phosphoric and sulphuric acids. This admixture of mixed acids contained 1% by weight of phosphoric acid and 1.5% by weight of sulphuric acid on dry biomass weight basis [total 2.5% acid on dry biomass weight basis]. The resultant mixture was then subjected to hydrolysis in a hydrolyser at a temperature of about 165° C. and a pressure of about 7 bar [absolute] for a period of 15 min at a pH of about 1.2. At the end of this pretreatment final slurry of about 550 kg contained about 20% total solids with about 13% of total insoluble solids. This slurry was then diluted with water to get about 16.4% total solids with about 10.4% of total insoluble solids. The pH of the diluted slurry was about 1.4. In the first step, about 3.5 kg magnesium oxide was added to said diluted slurry to achieve the pH of about 5.5. In this slurry, cellulose form about 55% of total insoluble solids. Then this slurry was subject to enzymatic hydrolysis by addition of about 30 mg of mix of celluloses per 1 g of cellulose present in the slurry. The enzymatic hydrolysis efficiency at the end of 120 h of hydrolysis at 50° C. was about 56% of theoretical maximum possible.

### EXAMPLE 9

A batch of about 130 kg bagasse having total solids around 92% by weight, of which cellulose is 36% by weight, hemicelluloses 21% by weight and insoluble lignin 19% by weight was used as a feedstock. It was subjected to mechanical shearing for size reduction to less than 40 mm particles affording about 120 kg dry particulate material. This particulate material was soaked in water for about 30 min. Then about 400 kg of slurry containing about 30% by weight total solids was prepared and continuously introduced into a plug-screw hydrolyser. Here the slurry was mixed with 160 L of the admixture of phosphoric and sulphuric acids. This admixture of mixed acids contained 1% by weight of phosphoric acid and 1.5% by weight of sulphuric acid on dry biomass weight basis [total 2.5% acid on dry biomass weight basis]. The resultant mixture was then subjected to hydrolysis in a hydrolyser at a temperature of about 165° C. and a pressure of about 7 bar [absolute] for a period of 15 min at a pH of about 1.2. At the end of this pretreatment, final slurry of about 545 kg contained about 20% total solids with about 13% of total insoluble solids. This slurry was then diluted with water to get 16.6%total solids with about 10.5% of total insoluble solids. The pH of the diluted slurry was about 1.4. In the first step about 2.8 kg magnesium oxide was added to said diluted slurry to achieve the pH of about 4.5. In the second step, about 2.75 kg sodium sulphite was added, at this stage pH raised to about 5.5. After these additions, the slurry was allowed to stand at 70° C. for about 30 min for effect of sulphite to take place on the insoluble solids present in said slurry. In this slurry, cellulose form about 55% of total insoluble solids. Then this sulphite treated slurry was subject to enzymatic hydrolysis by addition of about 30 mg of mix of cellulases per 1 g of cellulose present in the slurry. The enzymatic hydrolysis efficiency at the end of 120 h of hydrolysis at 50° C. was about 69.3% of theoretical maximum possible.

While the invention has been particularly shown and described with reference to embodiments listed in examples, it will be appreciated that several of the above disclosed and other features and functions, or alternatives thereof, may be desirably combined into many other different systems or applications. Also that various presently unforeseen and unanticipated alternatives, modifications, variations, or improvements therein may be subsequently made by those skilled in the art which are also intended to be encompassed by the following claims. Although the invention has been described with reference to specific preferred embodiments, it is not intended to be limited thereto, rather those having ordinary skill in the art will recognize that variations and modifications may be made therein which are within the spirit of the invention and within the scope of the claims.

1. A improved process for enzymatic hydrolysis of cellulosic biomass comprising:
(a) Providing a lignocellulosic material and preparing its mixture in water;
(b) pretreating said mixture with one or more acids at a desired temperature for a desired time period to obtain a first stream;
(c) adjusting pH of said first stream with a base to obtain a second stream;
(d) treating said second stream with sodium sulphite at a desired temperature for a desired time period to obtain a third stream;
(e) contacting said third stream with one or more of cellulolytic enzymes at desired temperature for a desired time period to obtain a sugar rich final stream; and
(f) subjecting said final stream to a fermenting yeast to obtain ethanol.

2. The process of claim 1, wherein said:
(a) acids comprises oxalic acid or sulphuric acid or phosphoric acid or a combination thereof;
(b) cellulolytic enzymes comprises one or more of cellulase and hemicellulase;
(c) enzymes are used between 10 mg and 100 mg per gram of cellulose present in said third stream;
(d) base is magnesium oxide;
(e) desired temperature to obtain said first stream ranges from about 140° C. to about 210° C.; and
(f) desired time period to obtain said first stream ranges from about 5 minutes to about 120 minutes.

3. The process of claim 1, wherein said desired temperature to obtain said third stream ranges from about 60° C. to about 80° C.

4. The process of claim 1, wherein said desired time period to obtain said third stream ranges from about 10 minutes to about 60 minutes.

5. The process of claim 1, wherein said desired temperature to obtain said final stream ranges from about 40° C. to about 80° C.

6. The process of claim 1, wherein said desired time period to obtain said final stream ranges from about 36 hours to about 120 hours.

7. The process of claim 1, wherein pH of said first stream is adjusted to between about 4 and about 5.

8. The process of claim 1, wherein the efficiency of enzymatic hydrolysis of cellulose is at least 50 percent of theoretical maximum.

9. The process of claim 1, wherein the efficiency of enzymatic hydrolysis of cellulose is increased up to about 15% after treatment by sodium sulphite.

10. The process of claim 1, wherein the amount of sodium sulphite or sodium carbonate used is about 0.5% to about 5% by weight of suspended total solids.
